(19) United States
(12) Patent Application Publication
(10) Pub. No.: US 2025/0009104 A1
(43) Pub. Date: Jan. 9, 2025

(54) SCALP APPLICATOR WITH HAIR-PREPARTING AND MISTING

(71) Applicant: SHENZHEN XINGOXYGEN TECHNOLOGY CO., LTD., SHENZHEN (CN)

(72) Inventor: DANNI WU, SHENZHEN (CN)

(73) Assignee: SHENZHEN XINGOXYGEN TECHNOLOGY CO., LTD., SHENZHEN (CN)

(21) Appl. No.: 18/762,037

(22) Filed: Jul. 2, 2024

(30) Foreign Application Priority Data
Jul. 5, 2023 (CN) 202310817938.1

(51) Int. Cl. A45D 24/28

(52) U.S. Cl. CPC A45D 24/28; A45D 2200/057

(57) ABSTRACT

The present disclosure relates to a scalp applicator and a hair-preparting comb unit. The scalp applicator has a main body that is convenient for a user to hold and operate with one hand, the scalp applicator further comprising a closed head end; a working end opposite the closed head end; a power button and an injection port arranged in the main body; and a hair-preparting comb unit detachably mounted at the working end, wherein the hair-preparting comb unit comprises two boat-shaped parts arranged side by side, wherein each of the two boat-shaped parts has a bow and a stem respectively at both ends, a gunwale, and a cabin, and a misting nozzle provided at the bottom of the cabin and configured to spray mist liquid during operation; and a hair-parting channel arranged between the adjacent sides of the two boat-shaped parts and separating the two boat-shaped parts.

### CROSS-REFERENCE TO RELATED APPLICATION

This application claims priority to Chinese invention application No. 202310817938.1, filed with China Patent Office on Jul. 5, 2023, entitled as “Scalp Applicator with Hair-preparting”, the content of which is incorporated herein by reference in its entirety.

### TECHNICAL FIELD

The present disclosure relates to the technical field of head health care, and more specifically, to a scalp applicator with hair-preparting and misting and a hair-preparting comb unit.

### BACKGROUND

With the development of society and the improvement of living standards, there are already technologies on the market for head health care, such as hair growth care, anti-hair loss treatment, scalp care, and so on. At present, the commonly used solution of the manufacturers is to apply medicine onto the scalp in the form of hair rollers, brushes, droppers, etc., such as the application of hair growth liquid or cream to try to treat or improve the alopecia, or the hair-loss problem. However, the existing solutions have various problems and shortcomings. The vast majority of products on the market use mechanical ball or roller to apply hair care fluid or medicine, and its construction of such kind of design is complex with cumbersome operation procedures, and there are many defects and deficiencies related to such design, such that its users are hard to adherence to the application, for example: the contact surface between the medicine application product of such design and the scalp is apt to cause discomfort to the users; the process of applying medicine purely relies on mechanical construction and its manual operation, the application of liquid medicine is not uniformly coated, and it is difficult for the user to control the dosage; during the application process, the applied liquid is often left in the hair before reaching the scalp, which wastes the liquid and adversely influences the effect of the treatment; the liquid is easy to remain after use and then cause blockage to the applicator; the construction of existing application devices are easy to pull hair during use, causing hair break or hair loss; and, the existing application devices are not ergonomic, inconvenient to use, and tends to induce poor user experience.

In view of the above listed disadvantages, there is a need in the present field for innovative, more practical and user-friendly scalp applicators and related technologies, in order to improve or eliminate the above defects, and achieve corresponding beneficial technical effects and more advantages.

The content included in the Related art of the present specification, including any references cited herein and any descriptions or discussions thereof, are incorporated only for the purpose of reference, thus shall not to be considered as limitation to the scope of the present disclosure.

### SUMMARY

In view of the above and other more concepts, the present disclosure is proposed.

In order to overcome the shortcomings of the prior art, one purpose of the present disclosure is to provide a scalp applicator with the function of hair-preparting, so as to solve the above technical problems and achieve more beneficial technical effects.

More specifically, according to the concept in one aspect of the present disclosure, a scalp applicator with hair-preparting and misting is provided. The scalp applicator having a main body that is convenient for a user to hold and operate with one hand, the main body having a generally rounded profile, the scalp applicator further comprising: a closed head end; a working end opposite the closed head end; a power button and an injection port arranged in the main body; and a hair-preparting comb unit detachably mounted at the working end, wherein the hair-preparting comb unit comprises: two boat-shaped parts arranged side by side, wherein each of the two boat-shaped parts has a bow and a stem respectively at both ends, a gunwale, and a cabin, and a misting nozzle provided at the bottom of the cabin; and a hair-parting channel arranged between the adjacent sides of the two boat-shaped parts and separating the two boat-shaped parts, wherein the misting nozzle is a pump-driven nozzle configured to spray mist liquid during operation.

According to an embodiment, a phototherapy device is provided at the working end or at the hair-preparting comb unit.

According to an embodiment, the misting nozzle is a pump-driven nozzle configured to spray mist liquid or to jet out atomized droplet during operation.

According to an embodiment, the bow has a rounded tip.

According to an embodiment, the bow and the stem of each boat-shaped part are symmetrical to each other.

According to an embodiment, the bow of each boat-shaped part, especially its tip, protrudes beyond the outer contour of the main body.

According to an embodiment, the misting nozzle is arranged at the bottom of the cabin.

According to an embodiment, the scalp applicator further comprises a base, wherein the base is constructed such that the main body is placeable onto the base in a standing manner when it is not in use.

According to an embodiment, the base has an overall convex contour presenting a saddle or an arch.

According to an embodiment, the base comprises two saddle-shaped portions corresponding respectively to the two boat-shaped parts of the hair-preparting comb unit, and a convex ridge for supporting the hair-parting channel thereon.

According to an embodiment, a pair of electrodes is provided at the bottom of the hair-parting channel, and a pair of electrodes is provided in the convex ridge at a position corresponding to the pair of electrodes at the hair-parting channel.

According to an embodiment, the main body has an outer profile of a generally rounded oblate column shape, wherein the power button and the injection port are arranged at both ends in a length extension of a cross-section of the oblate column shape, and wherein the longitudinal extension direction of the hair-preparting comb unit, the boat-shaped part and the hair-parting channel are consistent with the length extension direction; and wherein a liquid level window is provided at or near the injection port.

According to the concept in another aspect of the present disclosure, there is provided with a hair-preparting comb unit, comprising: at least one boat-shaped part, wherein the boat-shaped part has a bow and a stem respectively at both ends, a gunwale, and a cabin with a misting nozzle provided at the bottom of the cabin; and a hair-parting channel in the hair-preparting comb unit, arranged side by side with the gunwale of the boat-shaped part, wherein the misting nozzle is a pump-driven nozzle configured to spray mist liquid during operation.

According to an embodiment, the at least one boat-shaped part is a single boat-shaped part, and wherein a misting nozzle is provided at the bottom of the single boat-shaped part.

According to an embodiment, the at least one boat-shaped part is two boat-shaped parts arranged side by side, wherein a hair-parting channel is provided between adjacent gunwales of the two boat-shaped parts; and wherein a bottom of at least one of the two boat-shaped parts is provided with a misting nozzle.

According to an embodiment, the at least one boat-shaped part is three boat-shaped parts arranged side by side, wherein a hair-parting channel is provided between adjacent gunwales of each two adjacent boat-shaped parts; and wherein a bottom of at least one of the three boat-shaped parts is provided with a misting nozzle.

According to an embodiment, at least one of the bows has a rounded tip.

According to an embodiment, the bow and the stem of each of the boat-shaped parts have constructions symmetrical or asymmetrical to each other.

According to an embodiment, at least one of the hair-parting channels having a structure that is widened at both ends and narrowed or straight in the middle, or having a structure that is straight as a whole.

According to an embodiment, at least one of the hair-parting channels is provided with a through-hole for mounting an electrode therein.

According to an embodiment, comb teeth are provided on at least one gunwale of the hair-preparting comb unit.

According to an embodiment, at least one boat-shaped part of the hair-preparting comb unit is provided with two separated sub-cabins, and wherein a misting nozzle is provided at a bottom of at least one of the two sub-cabins.

According to an embodiment, at least one of the gunwales is flat or concave; and/or, at least one of the gunwales is convex in the middle section and concave in the two ends.

According to the concept in yet another aspect of the present disclosure, there is provided with a scalp applicator with hair-preparting and misting, wherein the scalp applicator has a main body, the main body comprising: a closed head end; a working end opposite to the closed head end; a power button and an injection port arranged in the main body; and an aforesaid hair-preparting comb unit, which is detachably mounted at the working end.

According to an embodiment, a liquid level window is provided at or near the injection port.

According to an embodiment, the liquid level window is provided with a liquid level scale line.

According to an embodiment, a lighting source such as an LED lamp may be added to the lower part of the liquid level window, i.e. near its bottom.

According to an embodiment, the electrode is of magnetic type.

According to one embodiment, the arc bending degree of the concave gunwale is suitable to fit the user's head shape.

Compared with the prior art, some of functions and technical effects according to one or more embodiment of the present disclosure may be listed as follows: (a) preparting, combing and massage: before applying the medicine, the hair is preparted through the scalp applicator to both sides of its boat-shaped part (s), exposing the parted hair gap and the scalp, convenient for applying the medicine thereto, and it is self-evident that the scalp applicator has the function of combing and massage; (b) spray application function: immediately after hair pre-parting, spray the atomized liquid into the parted hair gap and the scalp through the misting nozzle (s), so that the atomized liquid goes directly to the scalp; (c) automatic timing and dosage set function: in order to better meet the needs of users for different use dosages, the corresponding operation modes can be automatically set, such as by matching different scalp zones and the required time; (d) anti-clogging and draining design: after the liquid is sprayed under corresponding mode, a few seconds of air eject time is left to eject the residual liquid in the misting nozzle, and then the scalp applicator can be placed onto the specially designed base for draining, to achieve anti-clogging and draining functions; (e) replaceable hair-preparting comb unit: the hair-preparting comb unit can be detachable and replaceable, which is convenient for users to choose and replace the most suitable hair-preparting comb unit according to their own hair volume, head shape, hair length and so on, and the hair-preparting comb unit can be provided with combing or grate combing effect; (f) ergonomic design: through an overall oval (or elliptical) or rounded shape and a standing design of the scalp applicator, it is easy for users to hold and operate with one hand, with improved ergonomic design, user comfort, user friendliness, and easy access.

Further embodiments of the present disclosure may achieve other beneficial technical effects not listed individually, which may be partially described herein below and which are expectable and understood by a person skilled in the art after reading the present disclosure.

### BRIEF DESCRIPTION OF THE DRAWINGS

The above features and advantages and other features and advantages of these embodiments, and the implementation way thereof, would be apparent to those skilled in the art, the present disclosure and the embodiments thereof may be better understood, by reference to the following figures and the related description.

FIG. 1 is a schematic front view of the overall construction of a scalp applicator having a hair-preparting function according to an exemplary embodiment of the present disclosure.

FIG. 2 is a schematic side view of the scalp applicator in the embodiment shown in FIG. 1 from one side.

FIG. 3 is a schematic side view of the scalp applicator in the embodiment shown in FIG. 1, viewed from the other side opposite to FIG. 2.

FIG. 4 is a schematic perspective view of the scalp applicator in the embodiment shown in FIG. 1 in an inverted orientation, schematically illustrating the bottom construction and configuration of the working end of the scalp applicator (applicator' main body) engaged with the base.

FIG. 5 is a schematic view of the base of the scalp applicator in the embodiment shown in FIG. 1, schematically showing the main structure, functional features and construction of the base.

FIG. 6 is a separate perspective view of the hair-preparting comb unit of the scalp applicator as shown in FIGS. 1-5, schematically showing the overall construction of the hair-preparting comb unit after disassembly from the main body.

FIG. 7 is a schematic bottom view of the hair-preparting comb unit as shown in FIG. 6 in an inverted orientation, showing its bottom construction schematically.

FIG. 8 is a variant embodiment of the hair-preparting comb unit shown in FIG. 6, schematically showing a differently constructed gunwale of the boat-shaped part.

FIG. 9 is another variant embodiment of the hair-preparting comb unit shown in FIG. 6, schematically showing each boat-shaped part divided from the middle to form two separate sub-cabins.

FIG. 10 is yet another variant embodiment of the hair-preparting comb unit as shown in FIG. 6, schematically showing the construction of the hair-preparting comb unit with three boat-shaped parts arranged in parallel.

FIG. 11 is still another variant embodiment of the hair-preparting comb unit shown in FIG. 6, schematically showing the top construction (the bottom portion is not shown) of the hair-preparting comb unit with only a single boat-shaped part.

FIG. 12 is a schematic view of the bottom of the hair-preparting comb unit with a single boat-shaped part as shown in FIG. 11, schematically showing its bottom structure.

FIG. 13 is a schematic view of the bottom of a variant of the hair-preparting comb unit with a single boat-shaped part as shown in FIG. 11, schematically showing its bottom structure, in particular its one side or both sides with comb teeth.

### DETAILED DESCRIPTION

Details of one or more embodiments of the present disclosure will be set forth in the following description of the accompanying drawings and detailed description. Other features, objects, and advantages of the present disclosure may be apparent from these descriptions, drawings, and claims.

It should be understood that the illustrated and described embodiments are not limited in applications to the details of construction and arrangement of components set forth in the following description or illustrated in the accompanying drawings. The illustrated embodiments may be other embodiments and can be implemented or executed in various ways. The examples are provided by way of explanation of the disclosed embodiments and not by way of limitation. Indeed, it will be apparent to those skilled in the art that various modifications and variations can be made to the embodiments of the present disclosure without departing from the scope or spirit of the present disclosure. For example, features illustrated or described as part of one embodiment may be used with another embodiment to still produce additional embodiments. Accordingly, the present disclosure encompasses such modifications and variations that fall within the scope of the appended claims and their equivalents.

Also, it is to be understood that the wordings “comprising”, “including” or “having” and variations thereof used herein are for the purpose of description in an open manner and intended to contain items listed thereafter and equivalents thereof, as well as additional items if appropriate.

The present disclosure will be described in more detail below with reference to the attached drawings and specific embodiments. From these descriptions, drawings, and claims, other features, purposes and advantages of the present disclosure will be made clear.

### Embodiments of Scalp Applicator

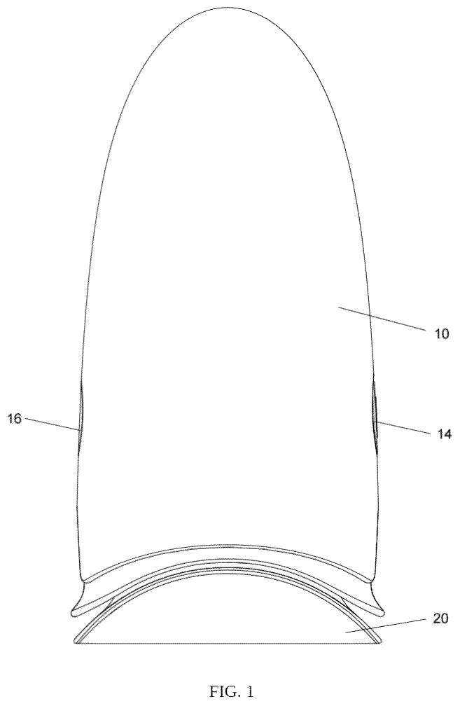

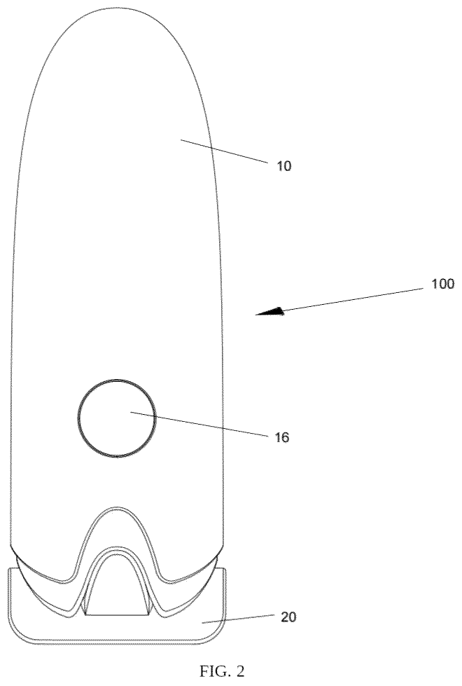

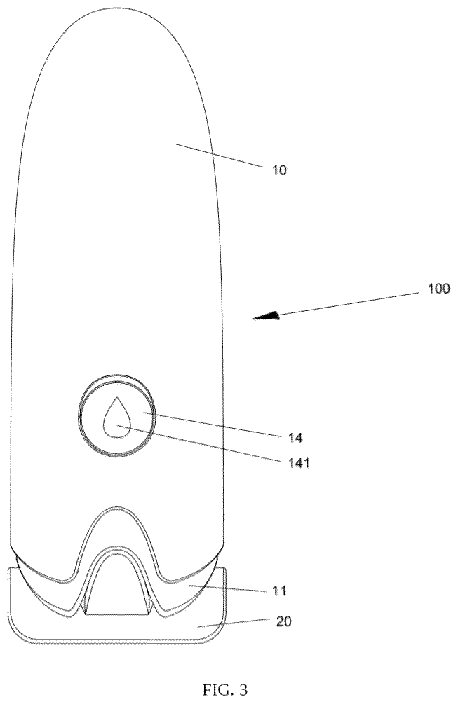

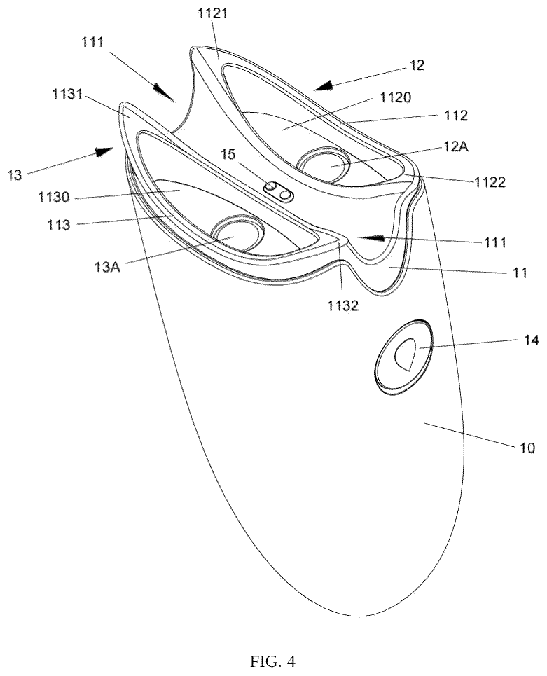

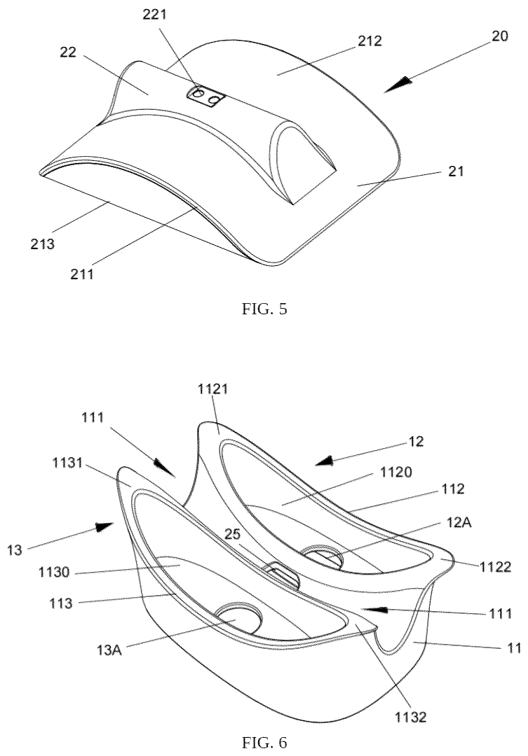

The main construction and configuration of a scalp applicator 100 according to an embodiment of the present disclosure are illustrated in FIG. 1-5. FIG. 1 is a schematic front view of the overall construction of a scalp applicator 100 having a hair-preparting function according to an exemplary embodiment of the present disclosure. FIG. 2 is a schematic side view of the scalp applicator 100 in the embodiment shown in FIG. 1 from one side. FIG. 3 is a schematic side view of the scalp applicator 100 in the embodiment shown in FIG. 1, viewed from the other side opposite to FIG. 2. FIG. 4 is a schematic perspective view of the scalp applicator 100 in the embodiment shown in FIG. 1 in an inverted orientation, schematically illustrating the bottom construction and configuration of the working end of the scalp applicator 100 (main body 10) engaged with the base 20. FIG. 5 is a schematic view of the base of the scalp applicator 100 in the embodiment shown in FIG. 1, schematically showing the main structure, functional features and construction of the base 20.

As shown schematically in FIG. 1-5, the scalp applicator 100 according to an embodiment of the present disclosure mainly comprises two portion, namely, a main body 10 of the scalp applicator, and a base 20.
main body 10 is the main function and operation part of the scalp applicator 100, which can be shaped as a rounded (or round) cylinder as a whole, which is not only for aesthetic appearance and safety, but also is easy to be held by the user during operation. When not in use, the main body 10 will be placed bottom down and engaged with the base 20 in the orientation shown in FIG. 1-2. The base 20 may be rechargeable or non-rechargeable, which is described in detail below. On the upper side of main body 10, as shown in FIG. 1-2, a round or rounded end is preferred. At the lower end of the main body 10 as shown in FIG. 1-2, that is, the working end, the main body 10 can be configured with hair parting combing features and the application (or applicator) features, which will be described in detail below. A liquid storage tank for the storage of applied liquid and a power supply such as battery (not shown) can be provided inside the main body 10.

As shown in FIG. 1-3, in the main body 10 and between the upper end and the lower end of the main body 10, and preferably on the two opposite sides of the main body 10 (for example, the left and right sides as shown in FIG. 1), a power button 16 and an injection port 14 are respectively arranged. The power button 16 is used to control the power-on and power-off of the scalp applicator 100 through manual touch or press. According to one example, a transparent or translucent liquid level window 141 can be optionally provided at the cover of the injection port 14 to display the liquid level in the liquid storage tank in real time, thus facilitating the timely addition and/or replacement of the formulation/medicine liquid when the liquid level is too low, and the liquid level window 141 can be optionally provided with liquid level marker (s) such as liquid level scales, etc. According to an example, the lower part of the liquid level window 141, that is, the part near its bottom, can be equipped with lighting source, such as LED light, and the matte effect can be additionally added near the bottom to soften the lighting effect, and the liquid level can be displayed in the liquid level window 141 to facilitate observation of the liquid level state. the injection port 14 and the power button 16 can be preferably positioned symmetrically, forming a visually stable look and an aesthetic effect. As a preferred example, the liquid level window 141 can be designed in appearance in a general shape of water drop, so that the user can intuitively understand its intrinsic function, and can facilitate visual observation of the liquid level of the liquid storage tank to know the amount of liquid remaining in real time. The injection port 14, preferably in the shape of a hole such as a round hole, has a relatively large size, such as a diameter sufficiently large to facilitate the user to align and insert the formulation/medicine liquid bottle into the injection port 14, in order to supplement/add the formulation/medicine liquid.

From the point of view of ergonomic design, as shown in FIG. 1-3, according to a preferred example, the arrangement of the injection port 14 and the power button 16 in the main body 10 is symmetrical, additionally, the position of the power button 16 (and the injection port 14) is designed such that when the user holds the main body 10 with his hand (the position of main body 10 during normal use is “upside down” to facilitate the user to hold in hand, as shown in FIG. 4), the index finger or thumb of the user during operation can naturally touch or press the power button 16. This design also ensures that the shape of the power button 16 is substantially corresponded with the normal-use position of main body 10 to improve user experience and avoid misoperation. In addition, the button size of the power button 16 can be designed large enough to further increase user friendliness and good touch feedback during operation.

According to a preferred example, the lower end of main body 10 as shown in FIG. 1-3, that is, the working end, can be provided with a hair-preparting comb unit 11, such as through plug-in fit in a removable manner. The detailed construction and configuration of the hair-preparting comb unit 11 is shown in more detail in FIG. 4. The hair-preparting comb unit 11 mainly plays the role of preparting the user's hair during use and preferably slightly ahead of the application of the formulation/medicine liquid, so that the preparting of hair exposes the scalp skin, facilitating the applicator nozzles 12A and 13A for applying formulation/medicine (or coating formulation/medicine) thereto; besides, the hair-preparting comb unit 11 can additionally play the role of massaging the scalp during use, and meanwhile may optionally comb hair, which will be described in detail below. In this respect, as an example, the applicator nozzle 12A, 13A may be a key part of a micro-misting system, i.e., a misting or atomizing nozzle, which may preferably be a pump-driven nozzle, configured to spray/discharge mist liquid or to jet out atomized formulation/medicine droplet which will be applied evenly and pervasively to the scalp during operation, as desired by the present invention. Particularly, the atomized droplet jet will help to more effectively apply the formulation/medicine, through the user's blocking hair (if any), to the user's scalp, especially if it is a higher speed one, in this regard, the scheme of air-pumping is preferred.

As illustrated schematically in FIG. 4, the working end of the main body 10 of the scalp applicator 100 according to an embodiment of the present disclosure is provided with the pre-dispensing comb 11, preferably in a removable fit manner. The hair-preparting comb unit 11, as shown in FIG. 4, can be installed at the working end of the main body 10 such as through snap-fit insertion, and also can be conveniently pulled out, for separate cleaning, maintenance and/or replacement. The hair-preparting comb unit 11 have two boat-shaped parts 12 and 13 arranged side by side and spaced apart, and the two boat-shaped parts 12 and 13 are so shaped and constructed as to preferably extend substantially along the length of the cross-section of the main body 10, and are spaced apart from each other so as to form a hair-parting channel 111 between the adjacent gunwales of the two boat-shaped parts 12 and 13, The hair-parting channel 111 is set for facilitating hair-parting during use and also for accommodating the parted hair passing there through. As shown in FIG. 4, a pair of electrodes 15 is optionally provided at the bottom of the hair-parting channel 111, so that the main body 10 can be charged through the base 20 engaging with the pair of electrodes 15. The hair-parting channel 111 is also constructed to fit with a raised contour of base 20, which is described in further detail below. According to a preferred example, the electrodes 15 are magnetic, i.e., formed from a magnetic conductive material, thus ensuring the stability and reliability of the engagement and electrical contact during charging.
boat-shaped parts 12 and 13 may preferably have substantially the same construction—they may of course have different construction, but the essential idea is largely the same. The boat-shaped part 12 is shaped like a small boat (schematically shown in FIGS. 1 and 4) and has a bow 1121 and a stem 1122 respectively at both ends, as well as a boat hull defining a gunwale 112 and a cabin 1120. The bow 1121 and the stem 1122 are preferably provided with round or rounded tips, so as to facilitate hair parting while in use, and meanwhile safe enough to avoid scalp scratches due to sharp (rather than rounded) tips otherwise. The boat-shaped part 12 preferably has a generally symmetrical construction at both ends, that is, the bow 1121 and the stem 1122 preferably have the same tip shape and being symmetrical to each other, so that the user can operate it for hair parting and applying from either end, i.e., from the bow 1121 or either from the stem 1122 end, without having to deliberately choosing the orientation of the boat-shaped part before use, thus further improving the user friendliness and user experience of the scalp applicator product. The bottom of the cabin 1120 can be designed with a through hole in which nozzle 12A can be arranged to apply formulation/medicine to the scalp (e.g. by spraying, atomizing, squeezing and painting, etc.), which can advantageously replace the solution of rolling and applying onto the hair and the scalp through roller.

Similarly, boat-shaped part 13 is also shaped like a small boat (schematically shown in FIGS. 1 and 4), with a bow 1131 and a stem 1132 respectively at both ends, and a boat hull defining a gunwale 113 and a cabin 1130. Bow 1131 and stem 1132 are preferably provided with round or rounded tips, so as to facilitate hair parting while in use, and meanwhile safe enough to avoid scalp scratches due to sharp (rather than rounded) tips otherwise. The boat-shaped part 13 also preferably has a generally symmetrical construction at both ends, that is, the bow 1131 and the stem 1132 preferably have the same tip shape and being symmetrical to each other, so that the user can operate it for hair parting and applying from either end, i.e., from the bow 1131 or either from the stem 1132 end, without having to deliberately choosing the orientation of the boat-shaped part before use, thus further improving the user friendliness and user experience of the scalp applicator product. The bottom of the cabin 1130 can be designed with a through hole in which nozzle 13A can be arranged to apply formulation/medicine to the scalp (e.g. by spraying, atomizing, squeezing and painting, etc.), which can advantageously replace the solution of rolling and applying onto the hair and the scalp through roller.

The boat-shaped parts 12 and 13 are preferably of substantially the same construction. A further advantage of this concept is that, the user experience is basically the same which can be attributed to the same and symmetrical construction of the boat-shaped parts 12 and 13 as well as the bow, no matter that the user holds the main body 10 by hand for application and/or hair-parting in the direction of the main body 10 as shown in FIG. 4, or reversely (i.e. rotating the main body 10 for 180 degrees so that the left and right positions of the main body 12 and 13 as shown are reversed).

As shown schematically in FIG. 1 and FIG. 4, the bows respectively at both ends of the boat-shaped part 12 and 13 are preferably protruded, that is, they are preferably protruded to a certain extent beyond the outline of the main body 10, as shown in FIG. 1. In this way, the contact surface of the hair-preparting comb unit 11 with the user's hair and scalp during the operation of scalp applicator 100 can be further increased, so as to facilitate more effective hair parting and liquid application.

The base 20 of the scalp applicator 100 of the present disclosure is further described in connection with the figures, in particular FIGS. 1 and 5.

As shown in FIG. 5, scalp applicator 100 can optionally be provided with base 20, for use with main body 10. Specifically, during periods of non-use, the main body 10 can be fitted onto the base 20. The underside of base 20 (not specifically shown) is generally flat to facilitate the smooth placement of base 20. The base 20 has a generally saddled (or arched) convex shape or contour that substantially matches the contour of the boat-shaped part of the hair-preparting comb unit 11, in particular, the convex contour of the base 20 matches the concave side profile and is compatible with the cabin. More specifically, the base 20 is provided with saddle 21 and a convex ridge 22 further projecting from saddle 21. The overall shape of the convex ridge 22 can be substantially matched with the hair-parting channel 111 and can accommodate and support the hair-parting channel 111, such as during charging. Accordingly, a pair of electrodes 221 in the convex ridge 22 may be provided at a position corresponding to a pair of electrodes 15 at the bottom of the hair-parting channel 111, and arranged to be electrically connected with the electrodes 15. The pair of electrodes 221 may also be magnetic. The saddle 21 comprises two saddle parts 212 and 213, whereby the convex ridge 22 is arranged between the two saddle parts 212 and 213 for the placement and support of the hair-parting channel 111 when the main body 10 is plugged in and onto the base 20. When the base 20 and the main body 10 are equipped with electrodes, the base 20 is the base further with charging function, and the battery in main body 10 can be charged via base 20 which can be electrically connected with an external power supply.

Another consideration and advantage of forming base 20 as a whole, and in particular saddle 21 and bulge 22, in a convex shape, is that base 20 facilitates and functions for quick liquid/water drying and draining of main body 10, which liquid/water may accumulate in the hair-parting channels 111, and in the cabins of boat-shaped parts 12 and 13. When the main body 10 is placed upside-down onto the base 20 with an overall convex contour, the residual liquid/water can be quickly drained, thus ensuring the hygiene and maintainability of the main body 10 after use.

As mentioned above, through holes can be provided at the bottom of cabins 1120 and 1130 to facilitate the placement of nozzle 12A for scalp application. And, for example, at the bottom of cabin 1120 and/or cabin 1130, a light therapy light source can be further designed and provided. The light therapy light source can be in the form of a red LED or an infrared ray light source such as an infrared LED, and the wavelength of the light emitted can be about 650 nm for phototherapy onto the scalp, or play an anti-inflammatory effect. The LED can be a single LED or an LED strip. The composition of the liquid formulation applied to the scalp depends on the needs of the user, as the liquid formulation in the liquid reservoir can be easily replaced. For example, the liquid formulation ingredient may be minoxidil tincture—which can be used to treat hair loss and alopecia areata, may be scalp care essentials, or a combination of several targeted formulation/medicinal ingredients, etc.

The following briefly describes the operation and the function of an embodiment of scalp applicator 100 for hair loss care. According to one example, the scalp applicator 100 may have three operating modes respectively corresponding to the dose and time of spray application to the hairline, half head, and full head.

Thus, an example scalp applicator 100 according to the present disclosure can be conveniently used by the user in different application scenarios, and can realize one or more of the following functions, among others.

Preparting, combing and massage: before applying the medicine, the hair is preparted through the scalp applicator to both sides of its boat-shaped part (s), exposing the parted hair gap and the scalp, convenient for applying the medicine thereto; and it is self-evident that the scalp applicator has the function of combing and massage.

Spray application function: immediately after hair pre-parting, spray the atomized liquid into the parted hair gap and the scalp through the misting nozzle (s), so that the atomized liquid goes directly to the scalp.

Automatic timing and dosage set function: in order to better meet the needs of users for different use dosages, the corresponding operation modes can be automatically set, such as by matching different scalp zones and the required time.

Anti-clogging and draining design: after the liquid is sprayed under corresponding mode, a few seconds of air eject time is left to eject the residual liquid in the misting nozzle, and then the scalp applicator can be placed onto the specially designed base for draining, to achieve anti-clogging and draining functions.

Replaceable hair-preparting comb unit: the hair-preparting comb unit can be detachable and replaceable, which is convenient for users to choose and replace the most suitable hair-preparting comb unit according to their own hair volume, head shape, hair length and so on.

Through the overall oval (oval) or rounded shape and standing design of the scalp applicator, it is easy for users to hold and operate with one hand, with improved ergonomic design, user comfort, user friendliness, and easy access.

Embodiments of hair-preparting comb unit that can be used with a head care device like scalp applicator

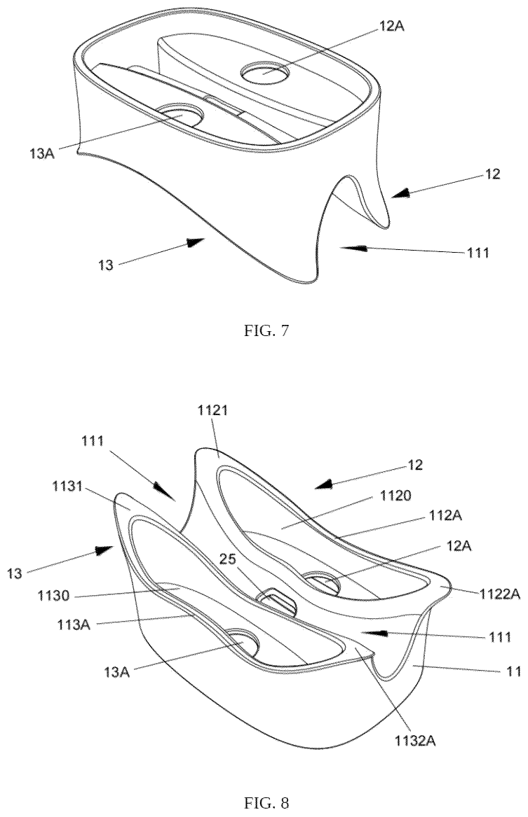

FIG. 6-7 is an overall schematic view of a hair-preparting comb unit 11 that can be used with a scalp applicator as shown in FIG. 1-5, schematically illustrating the overall construction of the individual hair-preparting comb unit 11 and its bottom construction. The hair-preparting comb unit 11 mainly plays the role of parting the user's hair during the use operation and plays the effect of hair preparting, and the scalp can be applied (or coated) with formulation/medicine liquid after the hair parting via misting nozzles 12A and 13A, the hair-preparting comb unit 11 can also play the role of massaging the scalp during the operation process. The description of the construction and design of the hair-preparting comb unit 11 shown in FIG. 6-7 may refer to the above-mentioned one, especially the description in connection with FIG. 4, so it will not be repeated here. In the bottom as shown FIG. 7, it can be seen that the mounting holes of nozzles 12A and 13A are through holes, so that nozzles 12A and 13A can connect and communicate with the relevant construction and arrangement inside the main body 10 of the scalp applicator. In addition, in the case that the scalp applicator is rechargeable, an electrode mounting hole 25 may optionally be provided as shown in FIG. 6 for mounting the electrode 15.

FIG. 8 is a variant embodiment of the hair-preparting comb unit 11 shown in FIG. 6, which schematically shows gunwales on boat sides of different structures of the boat-shaped part. The design of the rest of the embodiment is substantially similar to that shown in FIG. 6, so it is not necessary to go into details. As shown in FIG. 8, the gunwale on one side of boat-shaped part 13 is convex in the middle section, for example, while the gunwale on the other side of boat-shaped part 13 is concave (of course, it can be a normal gunwale shape, i.e. generally straight and flat). Similarly, the gunwale on one side of boat-shaped part 12 (not indicated) is convex, for example in the middle section, while the gunwale on the other side of boat-shaped part 12 can be concave (of course, it can be a normal gunwale shape, i.e. generally straight and flat). In addition, bow 1121 and stem 1122A of boat-shaped part 12 can be set to not be substantially symmetrical or identical in construction, for example, bow 1121 has a rounded tip and stem 1122A has a rounded blunt end. In FIG. 8, stem 1132A has a rounded tip substantially the same as bow 1131. Although not as shown in FIG. 8, bow 1131 and stem 1132A of boat-shaped part 13 can be set to be not substantially symmetrical or identical in construction, for example, bow 1131 is rounded with a rounded tip and stem 1132A is rounded with a relatively blunt end. In this way, for those who do not like the feel of irritation/hair entanglement when combing their hair with the hair-preparting comb unit of the present disclosure, they can choose to comb the hair with its rounded and relatively blunt end instead of a rounded tip to alleviate or avoid the feel of irritation/hair entanglement.

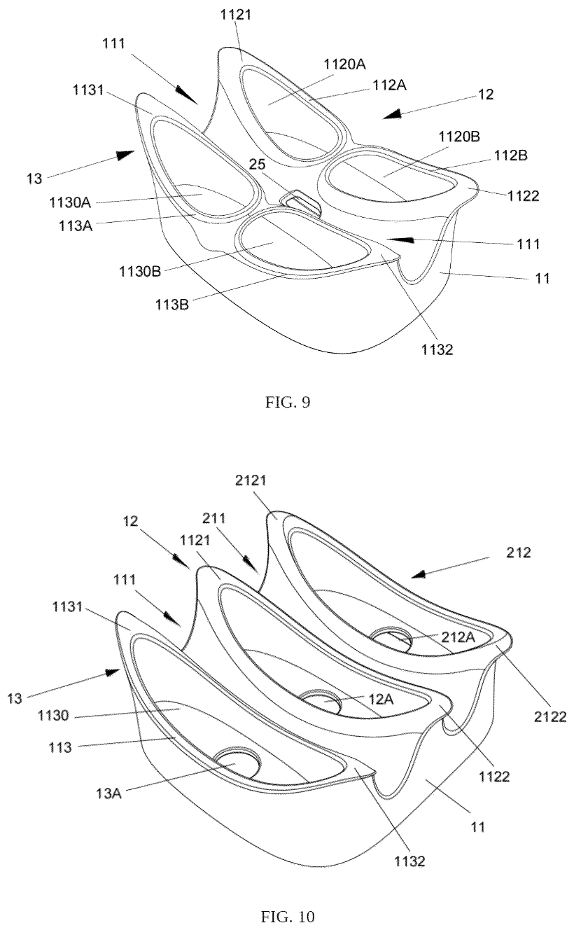

FIG. 9 is another variant embodiment of the hair-preparting comb unit 11 shown in FIG. 6, schematically showing each boat-shaped part 12, 13 separated from the middle to form two separate sub-cabins. The design of the rest of the embodiment is substantially similar to that shown in FIG. 6, so it is not necessary to go into details. As shown in FIG. 9, the boat-shaped part 12 is separated from the middle to form two separate sub-cabins 1120A and 1120B, and the gunwale on each side of the boat-shaped part 12 is preferably disconnected to form two separate gunwales, such as 112A and 112B. One concept of this design is that each sub-cabin 1120A, 1120B can be provided at the bottom with a misting nozzle (not shown in the figure) for applying formulation/medicine liquid, which misting nozzle can be respectively used to spray different formulation/medicine without interfering with each other, for example, one nozzle can be configured to spray baldness treatment agent, another nozzle can be configured to spray hair care agent, and so on. Similarly, boat-shaped part 13 can be separated in the middle to form two separate sub-cabins 1130A and 1130B, and the gunwale on each side of the boat-shaped part 13 is preferably disconnected to form two separate gunwales, such as 113A and 113B. Moreover, each sub-cabin 1130A, 1130B can be provided at the bottom with a misting nozzle (not shown in the figure) for applying different formulation/medicine liquid, without interfering with each other.

FIG. 10 is another variant embodiment of the hair-preparting comb unit 11 as shown in FIG. 6, schematically showing the construction of the hair-preparting comb unit 11 with three parallel arranged boat-shaped parts. The embodiment adds an additional parallel arranged boat-shaped part 212 based on the design and arrangement shown in FIG. 6. The design of the rest of the embodiment is substantially similar to that shown in FIG. 6, so it is not necessary to go into details. In particular, as shown in FIG. 10, boat-shaped part 212 may be design with the same construction as boat-shaped part 12 (although some details of its construction may be different), and a hair-parting channel 211 is formed between boat-shaped part 212 and boat-shaped part 12, which can also be used for hair parting, and so on. In addition, nozzle 212A can also be optionally arranged at the bottom of the sub-cabin/cabin of the boat 212, and so on.

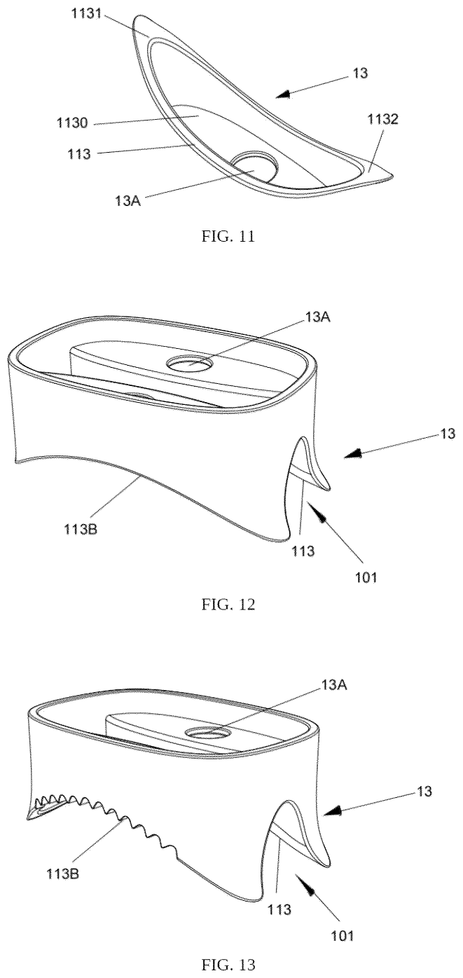

FIG. 11 is another variant embodiment of the hair-preparting comb unit 11 as shown in FIG. 6, showing schematically the construction of the hair-preparting comb unit 11 with only a single boat-shaped part 13 (only its top portion, with its main main body and bottom structure not drawn in this FIG. 11 for ease of presentation). FIG. 12 is a schematic bottom view of the hair-preparting comb unit with a single boat-shaped part 13 as shown in FIG. 11, schematically showing its bottom structure, and showing a hair-parting channel 101 being formed between the gunwale of the single boat-shaped part 13 of the hair-preparting comb unit 11 and the adjacent side/edge of the scalp applicator main body (rather than an adjacent gunwale of another boat-shaped part, since only one singe boat-shaped part provided in this embodiment as shown). The construction and design of the single boat-shaped part 13 shown in FIG. 11 may be identical to that of the boat-shaped part 13 of the hair-preparting comb unit 11 shown in FIG. 6, and thus no more details described herein. It is, of course, apparent and understandable to those skilled in the art that the single boat-shaped part 13 in this embodiment may also be other designs, such as the design of the boat-shaped part 12 or 13 in FIG. 8, or the design of the boat-shaped part 12 or 13 in FIG. 9, etc.

FIG. 13 is a schematic bottom view of a variant of the hair-preparting comb unit 11 with a single boat-shaped part shown in FIG. 11, schematically illustrating its bottom construction, particularly, wherein one side, preferably both sides (with one of which is a gunwale) of the hair-preparting comb unit 11 being provided with comb teeth 113B. Thus, such hair-preparting comb unit 11 can be used for hair parting/combing either longitude-wise (that is, in the longitudinal direction of the boat's body), or used for combing transversely (that is, transverse to the longitudinal direction of the boat-shaped part), that is, comb teeth 113B can be used to comb hair, wherein the comb teeth 113B can be arranged more finely than the currently shown in the figure, so that the hair-preparting comb unit 11 can be additionally used as a fine-toothed comb.

The terms “variant embodiment”, “another embodiment”, “another variant embodiment”, etc., as used in this specification, indicate that the features, structures, arrangements or characteristics described in said embodiment are intended to be incorporated in at least one other embodiment or example of the present disclosure. The foregoing description of the embodiment of the present disclosure is presented for purposes of illustration. The foregoing description is not intended to be exhaustive or to limit the present disclosure to the precise steps and/or forms as disclosed above, obviously, many modifications and variations can be made by the those skilled in the art in light of the above teachings. It is intended that the scope and its equivalents of the present disclosure will be defined by the appended claims.

What is claimed is:

1. A scalp applicator with hair-preparting and misting, the scalp applicator having a main body that is convenient for a user to hold and operate with one hand, the main body having a generally rounded profile, the scalp applicator further comprising:
a closed head end;
a working end opposite the closed head end;
a power button and an injection port arranged in the main body; and
a hair-preparting comb unit detachably mounted at the working end, wherein the hair-preparting comb unit comprises:
two boat-shaped parts arranged side by side, wherein each of the two boat-shaped parts has a bow and a stem respectively at both ends, a gunwale, and a cabin, and a misting nozzle provided at the bottom of the cabin;
a hair-parting channel arranged between the adjacent sides of the two boat-shaped parts and separating the two boat-shaped parts; and
wherein the misting nozzle is a pump-driven nozzle configured to spray mist liquid during operation.

2. The scalp applicator according to claim 1, wherein a phototherapy device is provided at the working end or at the hair-preparting comb unit.

3. The scalp applicator according to claim 1, wherein the bow has a rounded tip which protrudes beyond the outer contour of the main body.

4. The scalp applicator according to claim 1, wherein the bow and the stem of each boat-shaped part are symmetrical to each other.

5. The scalp applicator according to claim 1, wherein the scalp applicator further comprises a base, wherein the base is constructed such that the main body is placeable onto the base in a standing manner when it is not in use.

6. The scalp applicator according to claim 5, wherein the base has an overall convex contour presenting a saddle or an arch.

7. The scalp applicator according to claim 6, wherein the base comprises two saddle-shaped portions corresponding respectively to the two boat-shaped parts of the hair-preparting comb unit, and a convex ridge for supporting the hair-parting channel thereon.

8. The scalp applicator according to claim 7, wherein a pair of electrodes is provided at the bottom of the hair-parting channel, and a pair of electrodes is provided in the convex ridge at a position corresponding to the pair of electrodes at the hair-parting channel.

9. The scalp applicators according to claim 1, wherein the main body has an outer profile of a generally rounded oblate column shape, wherein the power button and the injection port are arranged at both ends in a length extension of a cross-section of the oblate column shape, and wherein the longitudinal extension direction of the hair-preparting comb unit, the boat-shaped part and the hair-parting channel are consistent with the length extension direction.

10. A hair-preparting comb unit, comprising:
at least one boat-shaped part, wherein the boat-shaped part has a bow and a stem respectively at both ends, a gunwale, and a cabin with a misting nozzle provided at the bottom of the cabin; and
a hair-parting channel in the hair-preparting comb unit, arranged side by side with the gunwale of the boat-shaped part;
wherein the misting nozzle is a pump-driven nozzle configured to spray mist liquid during operation.

11. The hair-preparting comb unit according to claim 10, wherein the at least one boat-shaped part is a single boat-shaped part.

12. The hair-preparting comb unit according to claim 10, wherein the at least one boat-shaped part is two boat-shaped parts arranged side by side, wherein a hair-parting channel is provided between adjacent gunwales of the two boat-shaped parts.

13. The hair-preparting comb unit according to claim 10, wherein the at least one boat-shaped part is three boat-shaped parts arranged side by side, wherein a hair-parting channel is provided between adjacent gunwales of each two adjacent boat-shaped parts.

14. The hair-preparting comb unit according to claim 10, wherein at least one of the bows has a rounded tip.

15. The hair-preparting comb unit according to claim 10, wherein at least one of the hair-parting channels having a structure that is widened at both ends and narrowed or straight in the middle, or alternatively having a structure that is straight as a whole.

16. The hair-preparting comb unit according to claim 10, wherein comb teeth are provided on at least one gunwale of the hair-preparting comb unit.

17. The hair-preparting comb unit according to claim 10, wherein the boat-shaped part of the hair-preparting comb unit is provided with two separated sub-cabins.

18. The hair-preparting comb unit according to claim 10, wherein at least one of the gunwales is generally flat or concave, or is convex in the middle section and concave in two ends.

19. A scalp applicator with hair-preparting and misting, wherein the scalp applicator has a main body, the main body comprising:
a closed head end;
a working end opposite to the closed head end;
a power button and an injection port arranged in the main body; and
a hair-preparting comb unit according to claim 10, which is detachably mounted at the working end.
